(19) United States
(12) Patent Application Publication
(10) Pub. No.: US 2006/0149378 A1
(43) Pub. Date: Jul. 6, 2006

(54) Articulating spinal disc prosthetic

(76) Inventors: Brad Chase, Ellicott City, MD (US); Kamran Aflatoon, Newport Beach, CA (US)

Correspondence Address: Royal W. Craig;Law Offices of Royal W. Craig, Suite 153, 10 N. Calvert Street, Baltimore, MD, 21202, US

(21) Appl. No.: 11/365,156

(22) Filed: Mar. 1, 2006

Related U.S. Application Data
(63) Continuation-in-part of application No. 10/997,823, filed on Nov. 24, 2004.
(60) Provisional application No. 60/657,755, filed on Mar. 2, 2005.

(51) Int. Cl. A61F 2/44

(52) U.S. Cl. 623/17.11

(57) ABSTRACT

A spinal disc prosthetic for replacement of cervical and/or lumber discs, that in each case allows a limited degree of rotational articulation, as well as durability and resistance to fatigue. The prosthetic is formed with three primary layers, including a superior (upper) plate, inferior (lower) plate, and intermediate layer, in a sandwich. The superior plate member is adapted to be secured on one side to an upper vertebra in a spinal column, and has a formed concave side exposed downwardly. The inferior plate member is adapted to be secured on one side to a lower vertebra in the spinal column, and has a substantially flat side exposed upwardly. The intermediate member is sandwiched between the superior and inferior plate members with conforming sides, and includes a short cylindrical post protruding downward into a circular recess in the inferior plate member to center it and to maintain a predetermined spacing there between. Two lateral pins on the intermediate member fit into two corresponding slots on the inferior member as guides and allow a degree of rotational freedom, and the post includes snap-in spring fingers that lock into the recess of the inferior member to prevent withdrawal.

### CROSS-REFERENCE TO RELATED APPLICATIONS

The present application derives priority from provisional patent application No. 60/657,755 filed Mar. 1, 2005, and is a continuation-in-part of U.S. patent application Ser. No. 10/997,823 filed Nov. 24, 2004.

### BACKGROUND OF THE INVENTION

1. Field of the Invention

The present invention relates to prosthetics and, more particularly, to prosthesis for use as a replacement for a lumbar and/or cervical disc in the spinal column of a human that allows both rotational and angular mobility.

2. Description of the Background

Intervertebral discs are subject to various forms of damage and degeneration due to mechanical stresses and aging. A variety of designs for artificial discs and disc prostheses have been proposed over the years. Cervical disc prosthetics pose a more difficult challenge in that the intervertebral spacing of the cervical vertebrae is smaller than for the lower vertebrae, etc., on the order of 8-9 mm for the cervical vertebrae, as compared with approximately 11 mm for the intervertebral discs. Moreover, the average age of patients undergoing spinal disc replacement is 42 years. Consequently, the life span of such a device should exceed 40 years. This requires an extremely high fatigue limit. Existing examples of disc prosthetics are shown in U.S. Pat. Nos. 6,517,580; 5,562,738; 5,676,701; 6,063,121; 6,162,252; 5,071,437; 5,522,899, and 6,132,465. While these designs purport to provide performance mimicking the function of the original disc, in practice, they do not articulate naturally and, consequently, do not function appropriately in the place of the original disc. Still other attempts have been made to replicate the natural action of both lumbar and cervical discs, including the following.

U.S. Pat. No. 4,759,766 to Buettner-Janz et al. (Humbolt University) issued Jul. 26, 1988 shows an intervertebral disc endoprosthesis with two end plates and a spacing piece, the spacing piece having opposing concave-convex surfaces with pins 13 that extend either into circular recesses 14, as shown in FIGS. 12 to 16, or into slot-shaped recesses 15, as shown in FIGS. 17 to 21.

U.S. Pat. No. 6,019,792 to Cauthen issued Feb. 1, 2000 shows an articulating spinal implant for intervertebral disc replacement that relies on an articulating ball-and-socket joint between two replacement disc elements that resists compression and lateral movement between the vertebrae, but allows pivotal movement, thereby preserving mobility. In this case the two replacement discs are hemispherical shapes.

U.S. Pat. No. 6,740,118 to Eisermann et al. (SDGI Holdings, Inc.) issued May 25, 2004 shows an intervertebral prosthetic joint with two plates formed with abutting convex and concave articular surfaces that cooperate to permit articulating motion. One of the articular surfaces has a surface depression that traps and allows removal of matter disposed between abutting portions of the articular surfaces.

U.S. Pat. No. 6,723,127 to Ralph et al. (Spine Core, Inc.) issued Apr. 20, 2004 shows an intervertebral disc that uses an intermediate “wave washer” between two plates that allows the plates to compress, rotate and angulate freely relative to one another, enabling the artificial disc to mimic a healthy natural intervertebral disc.

U.S. Pat. No. 5,401,269 to Buttner-Janz et al. (Waldemar Link) issued Mar. 28, 1995 shows an intervertebral disc endoprosthesis with two concave prosthesis plates sandwiching a convex prosthesis core. In one embodiment, the core has a nib (FIG. 8) that cooperates with at least one prosthesis plate to make possible a rotational movement.

United States Patent Application 20030040802 by Errico shows an artificial intervertebral disc having limited rotation using a captured ball and socket joint. The artificial disc has a pair of opposing baseplates, for seating against opposing vertebral bone surfaces. The base plates are separated by a ball and socket joint, the ball being secured by a post extending from one of the baseplates. The ball is captured within a socket formed in the other of the baseplates. The ball and socket joint therefore permits the baseplates to rotate relative to one another through a limited range and also angulate relative to one another.

United States Patent Application 20040158328 by Eisermann filed Aug. 12, 2004 shows a mobile bearing articulating disc with a plate having a concave recess, a second component having a second recess, and a projection adapted to engage the second recess surface to permit articulating motion between the first and second components. The projection 56 is shown to be a ball-and-socket type mechanism, with a notch 76 for removal of matter.

United States Patent Application 20040049280 by Cauthen filed Mar. 11, 2004 shows an articulating spinal implant for intervertebral disc replacement formed from three elements (see FIG. 5), two engaging adjacent vertebra. An articulating disc between the two elements resists compression and lateral movement between the vertebra, but allows the adjacent vertebra to articulate about an instantaneous axis of rotation.

United States Patent Application 20040176851 by Zubok et al. filed Sep. 9, 2004 shows a cervical disc replacement with first and second articulation plates having concave/convex surfaces sized and shaped to engage one another when the first and second members are disposed in the intervertebral disc space to enable the first and second vertebral bones to articulate in at least one of flexion, extension and lateral bending.

Although the above-described prosthetic discs as well as others have furthered technological development, none have fully solved the disc replacement problem. They pursue articulation, but lack durability and resistance to fatigue. It would be greatly advantageous to provide a fully-articulating spinal disc prosthetic having a high-wear capability and integrally-joined components that are extremely durable.

### SUMMARY OF THE INVENTION

Accordingly, it is an object of the present invention to provide a prosthetic disc with fully articulating capability, and also high-durability and resistance to fatigue.

It is another object to provide a fully-articulated prosthetic disc as above that makes use of integrally-joined components that cannot come apart.

In accordance with the foregoing object, the present invention comprises a lumbar and/or cervical disc prosthetic formed with three primary layers, including a superior (upper) plate, inferior (lower) plate, and intermediate layer, in a sandwiched configuration. The superior plate member is adapted to be secured on one side to an upper vertebra in a spinal column, and has a formed concave side exposed downwardly. The inferior plate member is adapted to be secured on one side to a lower vertebra in the spinal column, and has a substantially flat side exposed upwardly. The flat side is interrupted by a central cylindrical recess. The intermediate member has a convex side conforming to the concave side of the superior plate member, a flat downside conforming to the flat side of said inferior plate member, and is sandwiched between the superior and inferior plate members. The flat upside of the inferior member is interrupted by a circular recess. A short cylindrical post protrudes downward from the intermediate member and is seated inside the central recess of the inferior plate member to center it, lock it in place, and maintain a predetermined spacing there between. Both the post and the recess within which it resides have flat bearing surfaces for better wear. The post is coupled into the recess by a locking assembly that uses central snap-in spring fingers located at the bottom of the post as well. Additionally the locking assembly involves two lateral pins, one located at each end of the intermediate member, that fit into two corresponding slots located at each end of the inferior plate member. In this and equivalent configurations the locking assemblies prevent withdrawal of the intermediate member from the inferior plate member, thereby increasing reliability and durability. The particular configuration described herein limits rotational articulation to 20 degrees (10 degrees on each side), and also affords the durability and resistance to fatigue necessary for a 30-40 year lifetime.

### BRIEF DESCRIPTION OF THE DRAWINGS

Other objects, features, and advantages of the present invention will become more apparent from the following detailed description of the preferred embodiment and certain modifications thereof, in which

FIG. 1 is a side view of the spinal disc prosthesis 10 according to a preferred embodiment of the present invention.

FIG. 2A is a perspective view of intermediate member 108.

FIG. 2B is a side view of the intermediate member 108.

FIG. 3 is an exploded view of the spinal disc prosthesis 10 as in FIG. 1.

FIG. 4 is a perspective view of the intermediate member 108 as shown in FIGS. 1-3.

FIG. 5 is a side view of the intermediate member 108 as in FIG. 4.

FIG. 6 is a side view of the intermediate member 108 as in FIGS. 4-5 but rotated 90 degrees.

FIG. 7 is an exploded view of the cylindrical post 136 with central snap-in spring fingers 137 attached at the end of the post 136.

FIG. 8 is a perspective view of the inferior (lower) plate member 102 as shown in FIGS. 1 and 3.

FIG. 9 is a side view of the inferior (lower) plate member 102 as in FIG. 8.

FIG. 10 is a side view of the inferior (lower) plate member 102 as in FIGS. 8-9.

FIG. 11 is a top view of the inferior (lower) plate member 102 as in FIG. 10.

FIG. 12 is a perspective view of the superior (upper) plate member 100.

FIG. 13 is a top view of the superior (upper) plate member 100 as in FIG. 12.

FIG. 14 is a side view of the superior (upper) plate member 100 as in FIGS. 12 and 13 but rotated 90 degrees.

FIG. 15 is a side view of the superior (upper) plate member 100 as in FIGS. 12-14.

FIG. 16 is a sectional view of the view of the superior (upper) plate member 100 as in FIGS. 12-15.

### DETAILED DESCRIPTION OF THE PREFERRED EMBODIMENTS

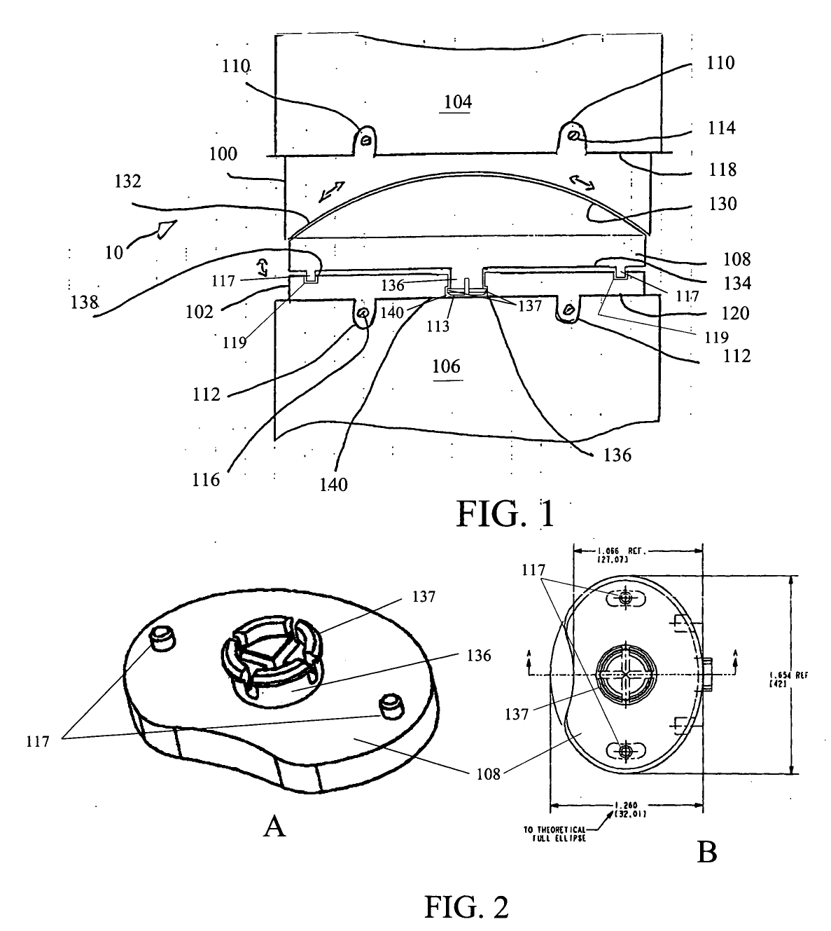

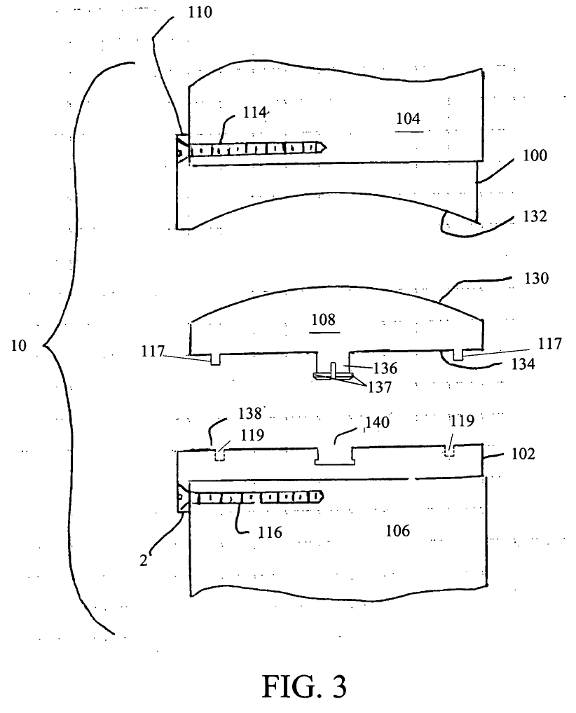

FIGS. 1 and 3 are a side view and exploded view, respectively, of the spinal disc prosthesis 10 (lumber or cervical) according to a preferred embodiment of the present invention. Referring to FIGS. 1 and 3, the spinal disc prosthesis 10 as illustrated includes an upper, or superior, plate member 100, and a lower, or inferior, plate member 102, which are adapted to be secured to upper and lower vertebra 104, 106, respectively, in a spinal column. An intermediate member 108 is provided, and is disposed between the upper and lower plate members 100, 102, once the prosthesis is assembled in the spinal column.

It is to be noted that the reference to the plate members as upper and lower members is for the purpose of identifying these members in the drawings. It may well be possible that the positions of the two plate members can be reversed.

Each of upper and lower plate members 100, 102 is provided with means for securement to the upper and lower vertebra. Many types of securement means are known in the art, and could be used with the present invention. For purposes of illustration, the plates 100, 102 are respectively provided with one or more tabs 110, 112 extending from the periphery of the plates 100, 102, and extending approximately laterally from the surface of the plates which will face the vertebra. As is known in the art, screws 114, 116 can be used to fasten the tabs to the vertebra 104, 106, through bores extending through the tabs. Plate members 100, 102 may further have their bone-contacting surfaces 118, 120 manufactured and/or treated or modified to facilitate or improve bonding to the bone. Again, several such approaches are known in the art and should be suitable for use with the present invention.

The upper plate member 100 preferably has a lower mating surface 132 that is concave and complementary in shape to the upper surface 130 of intermediate member 108. The intermediate member 108 is formed with a slightly smaller diameter than upper plate member 100, is sandwiched between the plate members 100, 102, and is formed with surfaces generally conforming to the opposing plate members 100, 102. Specifically, the intermediate member 108 is formed with a convex or domed upper surface 130 conforming to the concave lower mating surface 132 of the upper plate member 100. By providing such mating surfaces, the upper vertebrae 104 may shift either laterally or in a front or rearward direction, relative to the intermediate member 108 and lower vertebrae 106. The material or materials from which the intermediate member 108 is made, or the surface treatment thereof, can be selected to provide a desired degree of frictional engagement between the upper plate and intermediate member. While it is envisioned that the domed surface would preferably have a low coefficient of friction, it is possible to control or restrict movement between the upper plate 100 and intermediate member 108 by proper selection of the coefficient of friction of these two mating surfaces.

The lower surface 134 of intermediate member 108 is generally planar across the majority of the surface. The surface may, alternatively, be slightly convex. A post 136 protrudes from the lower surface 134. An upper mating surface 138 of lower plate member 102 is preferably generally planar, and has a recess 140 formed centrally therein to receive the post 136.

The post 136 and recess 140 are particularly configured for spacing the intermediate member 108 from the lower plate member 102, and for maintaining a rotating engagement therewith for purposes of the present invention. Thus, the recess 140 is generally cylindrical, conforming to the cylindrical post 136, with a conforming diameter and depth. This way, the intermediate member 108 is in complete contact with the top side of the lower plate member 102. If desired, the post 136 may be made slightly longer than the recess 140 in the lower plate member 102 with which it mates so as to allow for slight “flexion” of the intermediate member 108 (since the middle member will not be in “complete contact” with the bottom member). Generally, however, full contact is desired.

The post 136 remains free to rotate within recess 140 (allowing the intermediate member 108 to rotate as well), and the post 136 imposes a weight-bearing point of contact between the intermediate member 108 and upper mating surface 138 of lower plate member 102. This configuration presents a degree of freedom for rotation of the intermediate member 108 that more closely replicates that of a natural spinal joint. The post is preferably formed with a substantially planar face 113. The post 136 rests upon the flat face 113, and the latter serves as a bearing surface to support the intermediate member 108 and maintain an even keel, and yet to allow the foregoing articulation. This permits the relief of at least a portion of any compressive forces exerted on prosthesis 10 by the upper and lower vertebra, as well as permitting the upper and lower vertebra to rotate relative to each other through a small angle, as forces on the two vertebrae are transmitted to the prosthesis. The small circular and flat area of contact between post 136 and recess 140 also permits intermediate member 108 to rotate about a vertical axis relative to the lower plate 102, in the event that the spinal column experiences twisting forces. The upper and lower plate members 100, 102 are preferably made of a material that is compatible with the bone and, as noted previously, will preferably facilitate or promote bone grafting. The intermediate member 108 is preferably made of a material that is essentially non-resilient or of low resiliency, such as, for example, a metal, a ceramic, or a polymer having a low degree of resiliency. The function or operation of the prosthesis 10 in approximating the function or operation of a natural disc comes essentially from the degrees of freedom of movement provided between the upper and lower plates, and the intermediate member 108 disposed there between. The illustrated embodiment can be constructed with dimensions that are small enough to enable use of the prosthesis as a cervical disc prosthesis, where the intervertebral spacing is small, e.g., on the order of 8-9 mm. Thus, it is envisioned that the prosthesis 10 will be especially suitable for use in replacing damaged cervical discs. However, the prosthesis can be sized as required to serve as a lumbar disc prosthesis, as well.

The present invention also comprises a locking feature by which the post 136 is inserted into the recess 140 in such a way as to lock into it without detracting from the partial rotation capabilities described above. This may be accomplished with a variety of alternative and equally well-suited configurations, one example of which is described in detail herein.

The locking feature shown in FIGS. 1-3 entails forming the post 136 as a hollow annular member with side walls interrupted by a plurality of vertical notches, these notches defining snap-in spring fingers 137 located at the end of the post 136. The post 136 fits into the recess 140 via the snap-in spring fingers 137, the snap-in spring fingers 137 retracting a bit to allow insertion down past a slight lip in the recess 140 of lower plate member 100. As the snap-in spring fingers 137 clear the lip they spring outward in the recess 140 to lock the post 136 in place, and yet allow free rotation and pivoting. The locking engagement of the post 136 in recess 140 operates to maintain the intermediate member 108 in its desired position in the prosthesis assembly, and substantially prevents shifting of this element out of position.

FIG. 2A is a perspective view of intermediate member 108 with post 136, and FIG. 2B is a side enlarged view of the intermediate member 108 and post 136 located in the center. As can be seen in both FIGS. 2A-2B, the post 136 is formed with a distal lip or flange, and the side walls of hollow annular post 136 (including the lip) are interrupted by four evenly-spaced notches to define four snap-in spring fingers 137 located at the end of the cylindrical post 136.

In addition to the foregoing, the present invention also comprises a rotation-limiting feature to restrict relative rotation of the intermediate member 108 and lower plate member 100.

One example of an embodiment to accomplish this is best seen in FIGS. 2A & 2B, and comprises two flanking pins 117 protruding downwardly from the intermediate member 108 at opposite ends thereof. When the above-referenced post 136 is locked in place, the lateral pins 117 fit into two corresponding arcuate slots 119 located at each end of the inferior plate member 102. The slots 119 both comprise approximately 20 degrees of arc to give +/−10 degrees of rotation of the intermediate member 108 relative to the lower plate member 100 either clockwise of counterclockwise (10 degrees on each side).

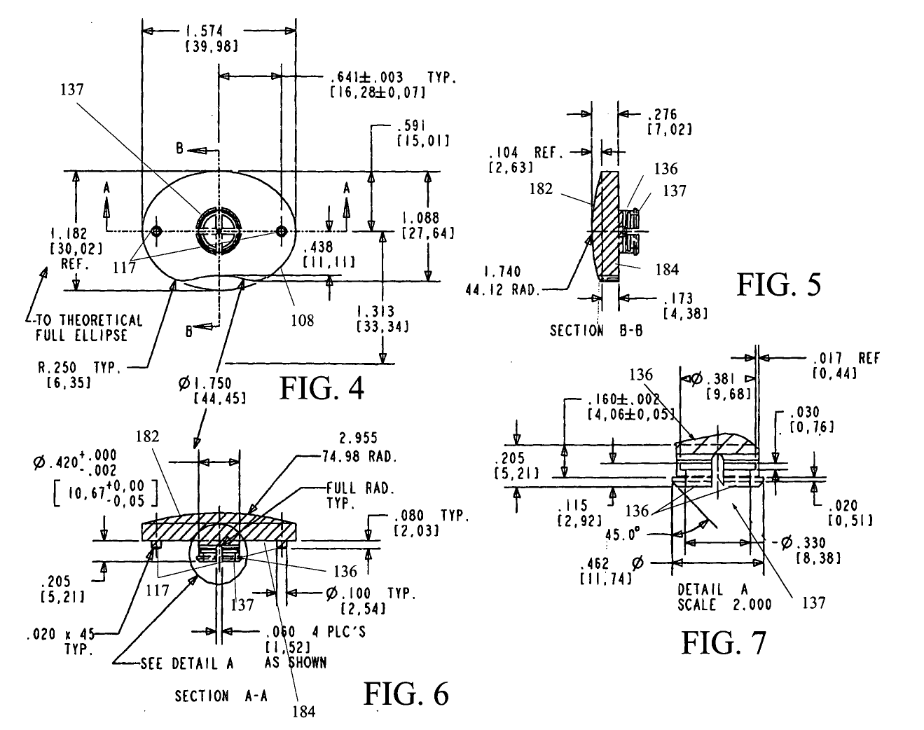

FIGS. 4-6 are a perspective view, side view, and a side rotated view (90 degrees), respectively, of the intermediate member 108 as shown in FIGS. 1-6 (using the locking feature of FIGS. 1-3) with an exemplary set of dimensions stated in millimeters. The intermediate member 108 is an oval-shaped disc having a convex top surface 182, a substantially flat bottom surface 184, and a downwardly protruding cylindrical post 136 extending centrally from the bottom surface 184. As seen in FIGS. 4-6, the central snap-in spring fingers 137 are located in the center of intermediate member 108. FIGS. 4 and 6 show two lateral pins 117 which are located at opposite ends of intermediate member 108. FIG. 7 is an exploded view of the central snap-in spring fingers 137 which are located at the bottom of post 136, and which snap into recess 140.

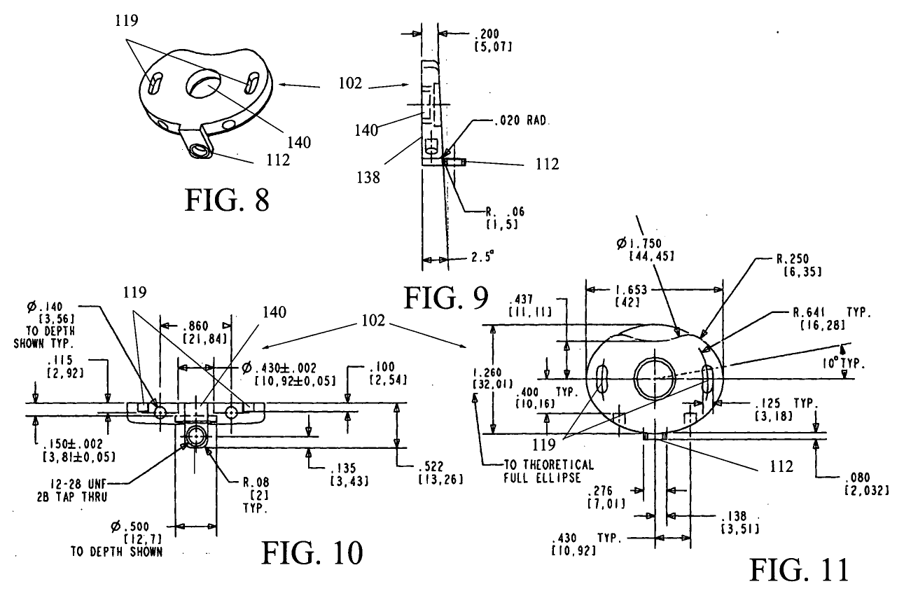

FIGS. 8-11 are a perspective view, side view, side view, and a top view, respectively, of the inferior (lower) plate member 102 (using the locking feature of FIGS. 1-3), with an exemplary set of dimensions stated in mm. The inferior (lower) plate member 102 is an oval-shaped disc having a substantially flat top surface 138, a slightly contoured bottom surface 139. The inferior plate member 102 is formed with one or more tabs 112 extending from the periphery orthogonally from the lower surface 139 to face the vertebra. As is known in the art, a screw can be used to fasten the tab(s) 112 to the vertebra through the bore-hole shown extending through the tab 112. The upper mating surface 138 of lower plate member 102 is generally planar, and has a recess 140 formed centrally therein to receive the post 136. The recess 140 is a cylindrical recess with a flat bottom surface. There are two slots 119 located on either side of the recess 140 of the lower plate member 102, to receive the two corresponding lateral pins 117 located on the intermediate member 108. The central snap-in spring fingers 137 at the end of the post 136, snap into the recess 140 and thus prevent the post 136 from being withdrawn. Therefore, the post 136 is inserted by a simple snap-in operation which locks the pins 117 therein, preventing inadvertent withdrawal.

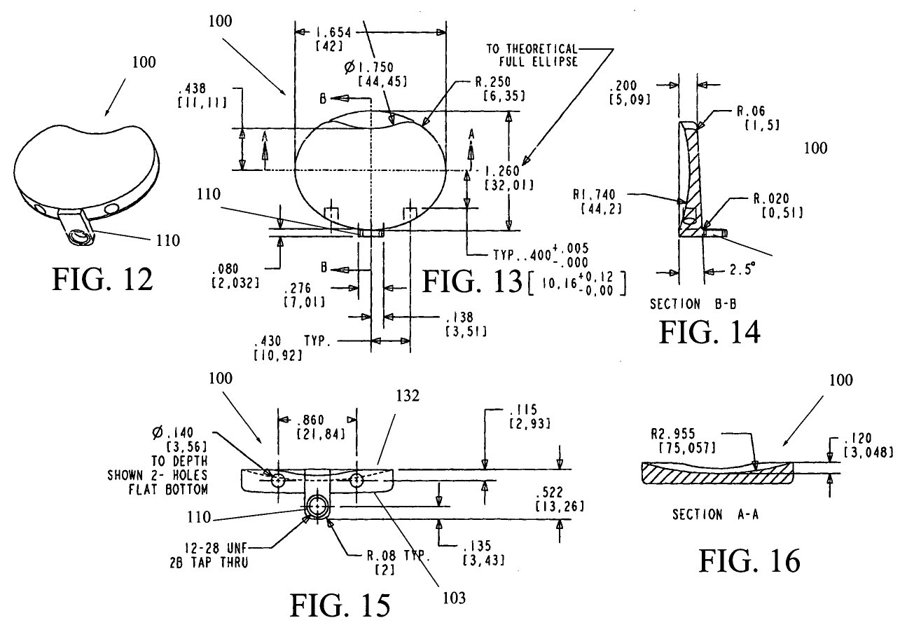

FIGS. 12-16 are a perspective view, top view, side rotated view (90 degrees), side view, and sectional view, respectively, of the superior (upper) plate member 100 with an exemplary set of dimensions stated in mm. The superior (upper) plate member 100 is an oval-shaped disc having a contoured top surface 103, and one or more tabs 110 extending from the periphery orthogonally from the upper surface 103 to face the vertebra. Again, a screw can be used to fasten the tab(s) 110 to the vertebra through the bore-hole shown extending through the tab 110. Upper plate member 100 preferably has a mating surface 132 which is concave, and which is complementary in shape to the domed surface 130 of intermediate member 108. By providing such mating surfaces, the upper vertebrae 104 may shift either laterally or in a front or rearward direction, relative to the intermediate member 108 and lower vertebrae 106.

Having now fully set forth the preferred embodiments and certain modifications of the concept underlying the present invention, various other embodiments as well as certain variations and modifications of the embodiments herein shown and described will obviously occur to those skilled in the art upon becoming familiar with said underlying concept. It is to be understood, therefore, that the invention may be practiced otherwise than as specifically set forth in the appended claims.

We claim:

1. A spinal disc prosthetic, comprising:
a superior plate member adapted to be secured on one side to upper vertebra in a spinal column, said superior plate member having another concave side;
an inferior plate member adapted to be secured on one side to a lower vertebra in a spinal column, said inferior plate member having an opposing substantially flat side interrupted by a central cylindrical recess; and
an intermediate member disposed between said superior and inferior plate members, said intermediate member having a convex side conforming to the concave side of the superior plate member, an opposing flat side conforming to the flat side of said inferior plate member, and a post extending centrally from the flat side and lockable inside the central recess of said inferior plate member to maintain a predetermined spacing there between.

2. The spinal disc prosthetic according to claim 1, wherein said cylindrical post has a flat face bearing surface for engaging a bottom of the central cylindrical recess in the inferior plate member.

3. The spinal disc prosthetic according to claim 1, wherein said cylindrical post has central snap-in spring fingers located at the end of said post which fits inside said recess to prevent withdrawal from inside said inferior plate member.

4. The spinal disc prosthetic according to claim 1, wherein said intermediate member has two lateral pins located at opposing sides of said intermediate member to prevent withdrawal of said intermediate member from inside said inferior plate member.

5. The spinal disc prosthetic according to claim 1, wherein said lateral pins and said central snap-in spring fingers insert into said inferior plate member allowing said lateral pins and said central snap-in spring fingers to lock said post in said recess.

6. The spinal disc prosthetic according to claim 1, further comprising a lumber disc prosthetic.

7. The spinal disc prosthetic according to claim 1, further comprising a cervical disc prosthetic.

8. A spinal disc prosthetic, comprising:
a superior plate member further comprising,
an upper surface having means for attaching said superior plate to an upper vertebra in a spinal column, and
a concave lower mating surface;
an inferior plate member further comprising,
an planar upper mating surface having a central cylindrical recess and two slots at opposite ends of said inferior plate member, and
a lower surface having means for attaching said inferior plate to a lower vertebra in a spinal column;
an intermediate plate member disposed between said superior and inferior plate members, said intermediate member further comprising,
a convex upper surface conforming to the concave lower mating surface of said superior plate member,
a planar lower surface conforming to the flat side of said inferior plate member,
two lateral flanking pins protruding downwardly at opposite ends of said intermediate member for insertion into said slots of said inferior plate member, and
a hollow annular post extending centrally from said planar lower surface, said post further comprising a distal lip and side walls interrupted by a plurality of vertical notches, said notches defining snap-in fingers at end of said post for snapping inside said recess of said inferior plate member.

9. The spinal disc prosthetic according to claim 8, wherein said upper surface of said superior plate member further comprises tabs extending from the periphery orthogonally of said upper surface, further wherein said tabs comprise bores for screw insertion therethrough to fasten said tabs to upper vertebra.

10. The spinal disc prosthetic according to claim 8, wherein said lower surface of said inferior plate member further comprises tabs extending from the periphery orthogonally of said lower surface, further wherein said tabs comprise bores for screw insertion therethrough to fasten said tabs to lower vertebra.

11. The spinal disc prosthetic according to claim 8, wherein said cylindrical recess has a smaller depth and larger diameter than said cylindrical post, thereby imposing clearance between said intermediate and inferior plate members and allowing said post to rotate within said recess.

12. The spinal disc prosthetic according to claim 8, wherein said slots are arcuate-shaped slots.

13. The spinal disc prosthetic according to claim 12, wherein said arcuate slots are defined by approximately 20 degrees of arc to give 10 degrees of rotation on each side of said intermediate plate member relative to said inferior plate member.

14. The spinal disc prosthetic according to claim 8, wherein said superior, inferior, and intermediate plate members are oval-shaped discs.

15. The spinal disc prosthetic according to claim 8, wherein said superior and inferior plate members are made of a bone-compatible material that will facilitate bone grafting.

16. A spinal disc prosthetic according to claim 8 wherein said intermediate plate member is made of material that is non-resilient or minimally resilient.

17. A spinal disc prosthetic according to claim 8 wherein said prosthetic is a cervical disc prosthetic.

18. A spinal disc prosthetic according to claim 8 wherein said prosthetic is a lumbar disc prosthetic.
